(19) United States
(12) Patent Application Publication
(10) Pub. No.: US 2008/0091155 A1
(43) Pub. Date: Apr. 17, 2008

(54) Condom catheter

(76) Inventor: Jean Paul Matter, Indian Hills, OH (US)

Correspondence Address: Wuilliam M. Selenke, 5 Drummiond St., Cincinnati, OH, 45328, US

(21) Appl. No.: 11/546,969

(22) Filed: Oct. 12, 2006

(51) Int. Cl. A61F 5/44

(52) U.S. Cl. 604/349; 604/352

(57) ABSTRACT

The bullet shaped condom urinary catheter has an air hose with a source of pressurized air ducted to near the larger open first end. The air hose terminates in an annular shaped hollow located near the larger open first end and the hollow is thinner on its inner circumference. That annular shaped hollow in the bullet shape condom has a series of pinholes to allow release of the air. The larger opening has of the condom has an inwardly projecting lip. An alternate embodiment of improved condom urinary catheter has an air hose which ducts air from an compressed air source and terminates in a series of pinholes in the inner wall to allow release of the air to flow from the larger end to flow out the urinary end. This improved condom urinary catheter has an inwardly projecting lip around said larger open first end and optionally adhesives to hold the condom on. Optionally, a garter or belt may mechanically retain the urinary catheter on the penis.

### FIELD OF INVENTION

The invention is concerned with improvements in urinary condom catheters, the so-called Texas condoms. More specifically, the present invention is concerned with providing a slow drying air flow between the inner wall of a urinary condom and the skin of the penis. Likewise, the present invention uses balloon like effects to hold the condom in place.

### BACKGROUND OF INVENTION

Many patients, especially older patients, through surgery, advanced age and so forth lose the ability to control micturition. Commonly, adult diapers are used. However, these adult diapers provide problems of their own. They are expensive and if they are not changed frequently the urine buildup can cause skin breakdown. In other instances, a Foley catheter is used to collect the urine. However, Foley catheters should be used only for short periods of time because a Foley provides a direct path for bacteria to induce bladder and urinary tract infections. One partial solution to urine control in male patients is the condom catheter. This device which rolled over the penis and retained in place by adhesives will allow the urine to drain from the urethra. However, condom catheters create a closed environment which is generally low in oxygen. This anoxic state, combined with corrosive characteristic of urine, can readily help induce skin breakdown followed by sores. Adhesives used to hold the condom catheter in place may prove to be an irritant to delicate aged skin, especially when the devices are being removed.

The improved urinary condom of the present invention addresses these two problems with a combined single solution. It provides pumped air to gently flow over the outer skin of the penis. This air flow provides several important benefits. First the constant low-level flow of air will aid the removal of the urine by blowing along on the inner wall of a condom catheter. Likewise, the air will provide a gentle drying effect to lessen the moisture content of the space between the condom catheter and the penis. Finally the air pump provides a constant atmospheric level of oxygen and in this inner space. An expansion hollow around the circumference of the condom catheter will provide a balloon-like means of anchoring the urinary condom to the penis.

### SUMMARY OF INVENTION

The invention is a bullet shaped condom urinary catheter with a larger open first end and a second end. The second end leads to a urine tube and an air hose is ducted to near the larger open first end. The air hose terminate it an annular shaped hollow around the bullet shaped condom which hollow is located near the larger open first end and the hollow is thinner on its inner circumference. That annular shaped hollow in the bullet shape condom has a series of pinholes to allow release of the air. The air hose is connected to an pressurized air source. The larger first opening has of the condom has an inwardly projecting lip.

An alternate embodiment of improved condom urinary catheter with a larger open first end and a second end that leads to a urine tube and has a retaining means. An air hose ducts air from an compressed air source and terminates in a series of pinholes in the inner wall to allow release of the air to flow toward the second end of the bullet shaped condom urinary catheter. This improved condom urinary catheter has an inwardly projecting lip around said larger open first end and retaining means such as adhesives. Optionally, a garter or belt may mechanically retain the urinary catheter on the penis.

### BRIEF DESCRIPTION OF FIGURES

FIG. 1 shows improved bullet shaped condom urinary catheter. FIG. 2 shows improved bullet shaped condom urinary catheter in longitudinal section. FIG. 3 shows cut A-A through improved bullet shaped condom urinary catheter as in FIG. 1. FIG. 4 shows an alternative embodiment of improved bullet shaped condom urinary catheter. FIG. 5 shows a cross section outline of the improved condom catheter in place over workpiece penis. FIG. 6 shows that a retaining means for urinary catheter may be a garter or belt.

### DETAILED DESCRIPTION OF FIGURES

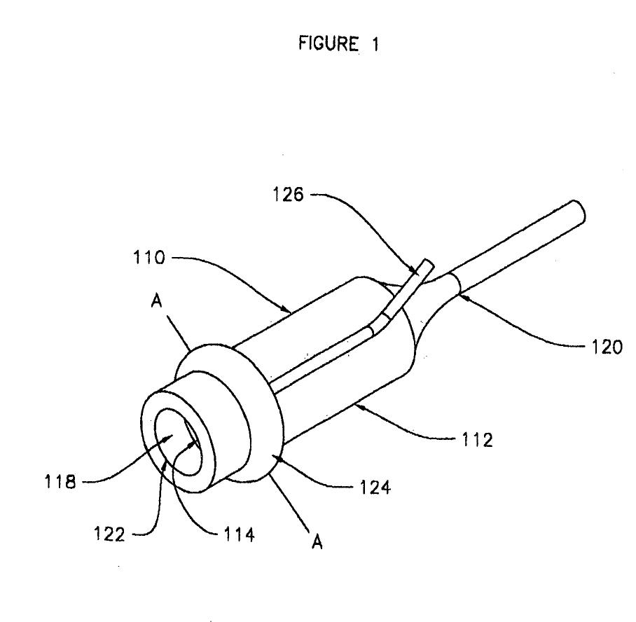

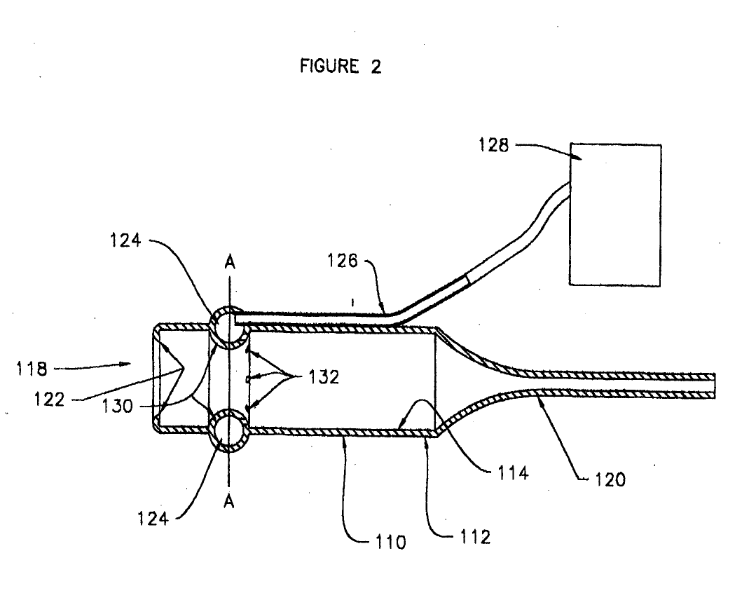

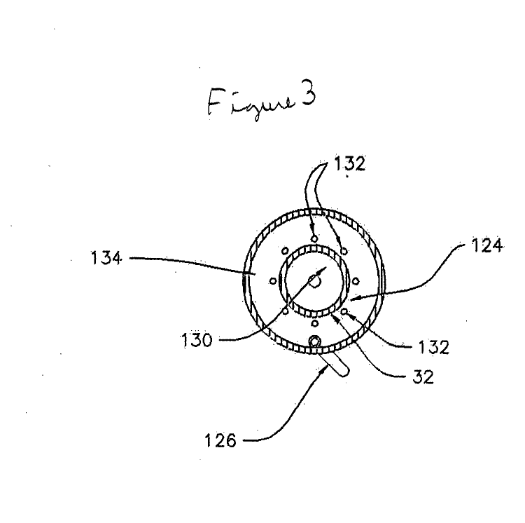

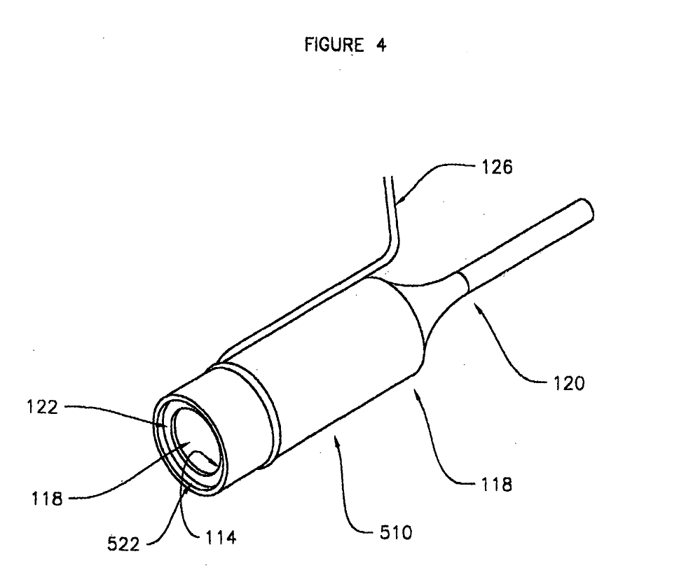

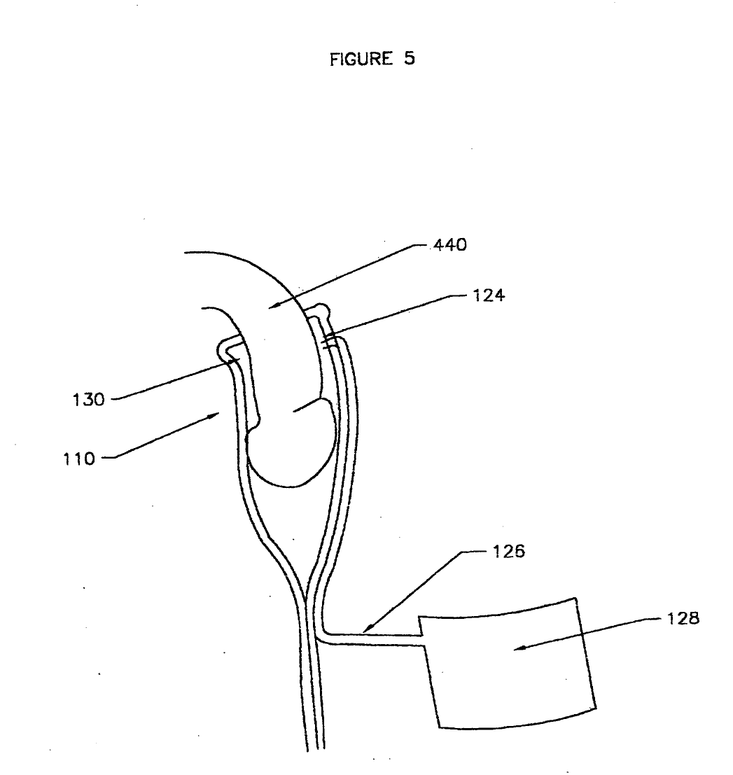

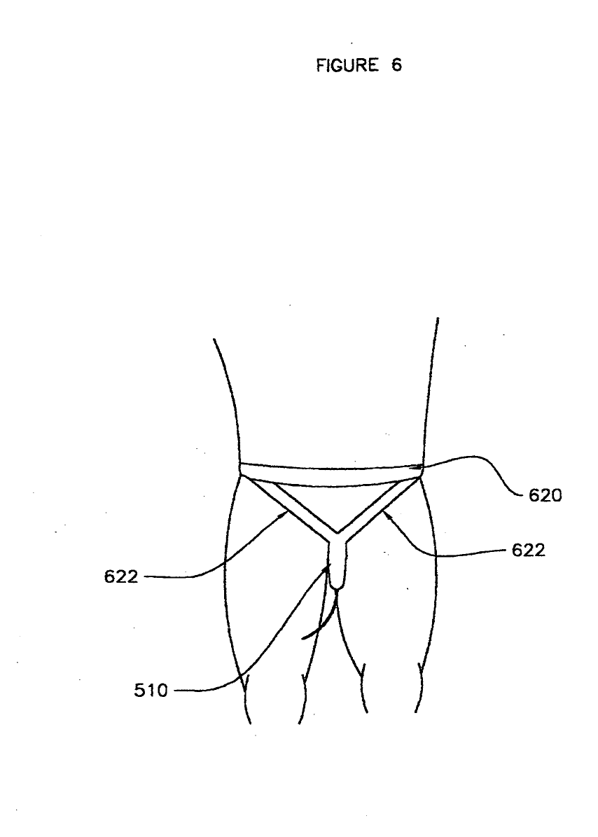

FIG. 1 shows improved bullet shaped condom urinary catheter 110 with an outer wall 112 and a larger open first end 118 which leads to the inner wall 114. Second end 120 leads to a urine tube collection system (not shown). Larger first open end 118 has and inwardly projecting annular lip 122. Air hose 126 originates in generic workpiece air pump 128 (not shown) and terminates in annular shaped hollow 124. This flowing air exits annular shaped hollow through pinholes in inner wall 114 and acts as a prophylactic to prevent the bullet shaped Texas condom from causing skin breakdown by removing the urine and keeping the outer surface of the patient's penis relatively dry.

FIG. 2 shows in longitudinal section improved bullet shaped condom urinary catheter of FIG. 1. Improved bullet shaped condom urinary catheter 110 has an outer wall 112 and an inner wall 114 with a larger open first end 118. Second end 120 leads to a urine tube (not shown). Larger first open end 118 has an inwardly projecting annular lip 122. Shown in cross section is an annular shaped hollow 124. Terminating in annular shape hollow 124 is air hose 126. Air hose 126 originates in generic workpiece air pump 128 or another pressurized air source. Air from workpiece air pump 128 inflate its annular shapes hollow 124 which expands preferably in an inwardly way because the thin inner wall 130 is more elastometric than the other circumference of wall section of angular shape hollow 124. Air that inflates angular shaped hollow 124 exits through a series of pinholes 132 to provide a constant flow of air through the bullet shaped Texas condom to aid the removal of the urine and help keep the outer surface of the patient's penis relatively dry.

FIG. 3 shows cut A-A through annular shape hollow 124 as in FIG. 2. Air hose 126 originates in generic workpiece air pump 128. Air hose 126 inflate annular shapes hollow 124 which expands preferably in an inwardly way because the thin inner wall 130 is more elastometric than the remainder of circumference of wall 134 of angular shape hollow 124. Air from expanded annularly shape hollow 124 escapes through a series of pinholes 132.

FIG. 4 shows an alternative embodiment of improved bullet shaped condom urinary catheter 510 with an outer wall 112 and a larger open first end 118. Second end 120 leads to a urine tube (not shown). Larger first open end 118 has and inwardly projecting annular lip 122 as a retaining means to hold the device in place on the penis. A first alternate means of holding the catheter in place is with adhesives 522 on the inner surface 114 of condom 510. A second alternative means is a garter or belt-like device to mechanically hold the urinary condom in place over the penis. Air hose 126 originates in generic workpiece air pump 128 (not shown). Air hose 126 terminates in an annular ring with a series of pinholes through the inner surface 114. The air exits through second end 120 and urine tube. This flowing air acts as a prophylactic to prevent the bullet shaped Texas condom from causing skin breakdown by removing the urine and keeping the outer surface of the patient's penis relatively dry.

FIG. 5 shows a cross section outline of the improved condom catheter 510 in place over workpiece penis 440. Air hose 126 terminates in an annular ring with a series of pinholes through the inner surface 114 of condom 510. Air hose 126 originates in generic workpiece air pump 128 which provides the air to flow over the penis to maintain a constant atmospheric level of oxygen. It is within the scope of the present invention that the second end 120 may terminate in a source of gentle suction.

FIG. 6 shown an alternative means other than adhesives to hold the urinary condom in place. A belt 620 has garter extensions 622 which attach to the urinary condom 510.

I claim:

1) An improved condom urinary catheter comprising:
a bullet shaped condom urinary catheter with a larger open first end and a second end;
said second end leads to a urine tube;
an air hose ducted to near said larger open first end;
said air hose terminate it an annular shaped hollow around said bullet shaped condom;
said annular shaped hollow in said bullet shape condom has a series of pinholes to allow release of the air toward said second end of said tube.

2) An improved condom urinary catheter as in claim 1) wherein said air hose is connected to an pressurized air source.

3) An improved condom urinary catheter as in claim 1) wherein said annular shaped hollow is thin on its inner circumference.

4) An improved condom urinary catheter as in claim 1) wherein said larger first opening has and inwardly projecting lip.

5) An improved condom urinary catheter comprising:
a bullet shaped condom urinary catheter with a larger open first end and a second end;
said second end leads to a urine tube;
a retaining means;
an air hose ducted to near said the larger open first end
said air hose terminates in a series of pinholes to allow release of the air to flow toward said second end of said bullet shaped condom urinary catheter.

6) An improved condom urinary catheter as in claim 5) wherein said retaining means is an inwardly projecting lip around said larger open first end.

7) An improved condom urinary catheter as in claim 5) wherein said retaining means is adhesives.

8) An improved condom urinary catheter as in claim 5) wherein said retaining means is a garter or belt mechanically retaining said urinary catheter
